(19) United States
(12) Patent Application Publication
(10) Pub. No.: US 2026/0224810 A1
(43) Pub. Date: Aug. 6, 2026

(54) SYSTEM AND METHOD FOR DETECTING OVER-INFUSION DUE TO MECHANICAL FAILURE OF AN INFUSION PUMP

(71) Applicant: CAREFUSION 303, INC., San Diego, CA (US)

(72) Inventors: Richard Stor WU, San Diego, CA (US); Joseph H. GRUCHACZ, San Diego, CA (US); Jay DAVE, Carlsbad, CA (US)

(21) Appl. No.: 19/153,932

(22) PCT Filed: Feb. 7, 2023
(86) PCT No.: PCT/US2023/012549

§ 371 Date: Aug. 5, 2025

(51) Int. Cl. A61M 5/168; A61M 5/142

(52) U.S. Cl. CPC A61M 5/16854; A61M 5/14228; A61M 2205/18; A61M 2205/583

(57) ABSTRACT

An apparatus, method, and system for detecting an over-infusion of a fluid due to a mechanical failure of an infusion pump. A pressure sensor of an infusion pump measures a pressure of a fluid in an infusion line loaded into the pump over at least a portion of a pumping cycle. The pumping cycle includes application of at least one occluder and at least one pumping finger to the infusion line to move the fluid through the infusion line. A control unit associated with the pump determines a metric based on measuring the pressure over the at least the portion of the pumping cycle, determines whether the metric satisfies a fault threshold corresponding to a pump fault and, responsive to the determination, provides an alert indicating the pump fault and terminates or reduces the pumping of the fluid through the infusion line.

### BACKGROUND

Large volume pumps (LVPs) can pump large fluid volumes (e.g., pumps delivering 100 mL or more of fluid from a single container) and usually operate through peristalsis, where a rotor turning across a section of tubing causes fluid to move through the tubing through positive displacement. LVPs may be subject to maintenance for various failures over time. One potential mode of failure includes over infusion. For example, occluders that pinch the tubing to prevent free flow may under-compress the tubing or fail, causing excess fluid to be infused into a patient. In some instances, the pump may infuse faster than the programmed flow rate (more rapidly than intended). In such case, a clinician may revisit a patient after a period of time only to discover an empty bag when it would have been otherwise expected to be half full. While the clinician would immediately suspect a mechanical defect, the defect is often not discovered until it is too late. Currently there are no adequate test systems, devices, or methods to identify mechanical failures that lead to over infusion quickly and efficiently.

### SUMMARY

There is a need to verify the proper operation of LVP infusion pumps with regards to over-infusion due to mechanical failures in pumps with multiple occlusion valves and fingers. Accordingly, the subject technology provides an apparatus and method for efficiently identifying occluder valves that are leaking.

According to various implementations, a system for detecting an over-infusion of a fluid comprises: an infusion pump; an pressure sensor disposed within the infusion pump to measure pressure of a fluid in an infusion line loaded into the infusion pump; and a control unit configured to: measure, over at least a portion of a pumping cycle of the infusion pump, with the pressure sensor, a pressure of the fluid in the infusion line loaded into the infusion pump, the pumping cycle including application of at least one occluder and at least one pumping finger to the infusion line to move the fluid through the infusion line; determine a metric based on measuring the pressure over the at least the portion of the pumping cycle; determine that the metric corresponds to a fault threshold associated with the at least the portion of the pumping cycle corresponding to an over-infusion fault; and provide, for display on a display device associated with the infusion pump, an alert indicating the over-infusion fault.

A method for detecting an over-infusion of a fluid comprises: measuring, over at least a portion of a pumping cycle of an infusion pump, with a pressure sensor, a pressure of the fluid in the infusion line loaded into the infusion pump, the pumping cycle including application of at least one occluder and at least one pumping finger to the infusion line to move the fluid through the infusion line; determining a metric based on measuring the pressure over the at least the portion of the pumping cycle; determining that the metric corresponds to a fault threshold associated with the at least the portion of the pumping cycle corresponding to an over-infusion fault; and providing, for display on a display device associated with the infusion pump, an alert indicating the over-infusion fault. Other aspects include corresponding apparatus (e.g., an infusion device), systems and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts a perspective view of an example infusion device showing an infusion set in place within the infusion device, according to various aspects of the subject technology.

FIG. 1B depicts an example patient care unit shown, according to various aspects of the subject technology.

FIG. 2 depicts an example pumping mechanism of an infusion pump including two occluder valves, according to various aspects of the subject technology.

FIG. 3 depicts an example occluder valve, including an occluder element, according to various aspects of the subject technology.

FIG. 4 depicts an example camshaft of a pumping element, according to various aspects of the subject technology.

FIG. 5 depicts an example cam phase diagram for testing a pumping element, according to various aspects of the subject technology.

FIG. 6 depicts example fill and delivery phases, according to various aspects of the subject technology.

FIG. 7 depicts fault profiles in an upstream pressure response of a mechanically failing pumping mechanism, according to various aspects of the subject technology.

FIG. 8A depicts a first example process for testing a pumping element, according to aspects of the subject technology.

FIG. 8B depicts a second example process for testing a pumping element, according to aspects of the subject technology.

FIG. 9 is a conceptual diagram illustrating an example electronic system for detecting an over-infusion of a fluid, according to aspects of the subject technology.

### DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

According to various implementations of the subject technology, a fluid tubing is loaded in an infusion pump and primed with a fluid. Upper and lower occluder elements of an infusion pump are activated to compress the fluid tubing to isolate the fluid in an upstream portion of the tubing from a downstream portion of the tubing. While the fluid tubing is compressed by the occluder elements, a pumping element of the infusion pump is activated to compress an intermediate portion of the tubing, between the downstream portion and the upstream portion, to cause a pressure increase within the fluid tubing. The lower occluder is opened and the fluid flows downstream.

As will be described further, infusion pumps rely on precise timing of the foregoing compression mechanisms on a fluid tubing to deliver fluid to a patient. Mechanical failure of these compression mechanisms may lead to conditions wherein the fluid free flows into the patient at a greater rate than desired. For example, the purpose of an occluder valve is to temporarily block the fluid flow to maintain a given flow rate. If there is not enough force (e.g., due to a broken spring) or the gap distance is too wide (e.g., due to material such as clothing or wires caught in door) then the tube may remain open and fluid will flow. Using at least one pressure sensor, a determination may be made as to whether the occluder is functioning properly based on a pressure response in a portion of the fluid tubing on a side of an activated occluder element, opposite the intermediate portion of the tubing.

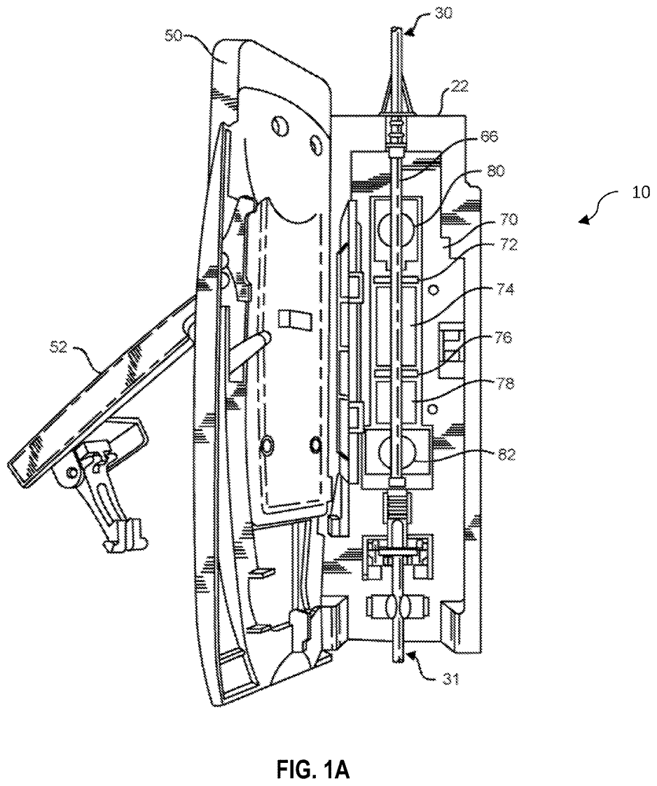

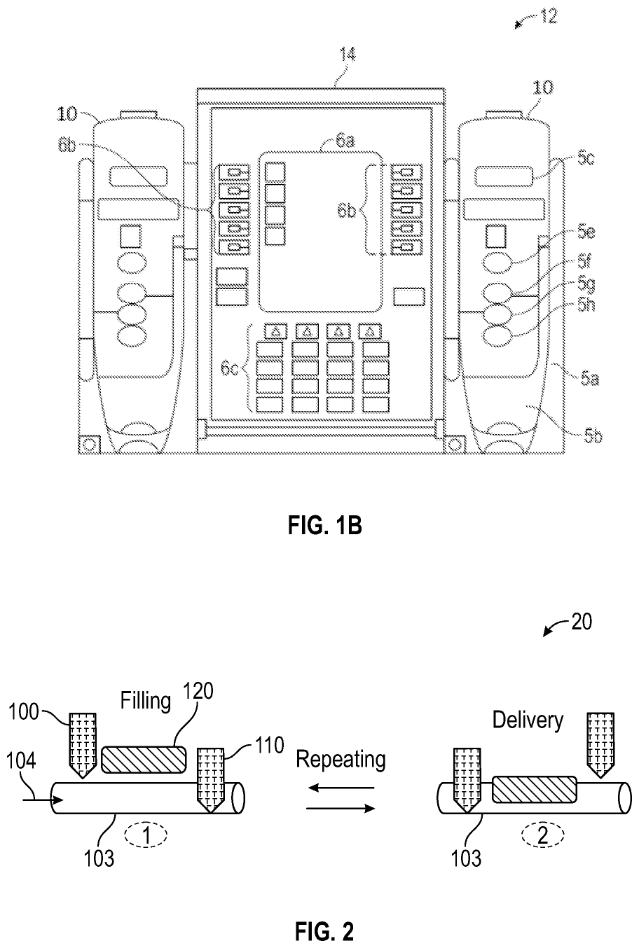

FIG. 1A depicts a perspective view of an example infusion device showing an infusion set in place within the infusion device, according to various aspects of the subject technology. An infusion system for parenteral infusion of a medical fluid to a patient comprises a pump unit, a major part of which comprises a housing which accommodates, in manner known per se, a cam system (not shown) controlling a plurality of fingers of a pumping mechanism, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls, all in manner known per se.

As shown in FIG. 1A, an infusion pump 10 is shown in perspective view with the front door 50 open, showing the upstream fluid line 30 (e.g., portion between fluid container and the infusion pump 30) and downstream fluid line 31 (e.g., portion between the infusion pump 31 and a patient) in operative engagement with the infusion pump 10. The infusion pump 10 directly acts on a tube 66 that connects the upstream fluid line 30 to the downstream fluid line 31 to form a continuous fluid conduit, extending from a respective fluid supply to a patient, through which fluid is acted upon by the pump to move fluid downstream to the patient. Specifically, a pumping mechanism 70 acts as the flow control device of the pump to move fluid though the conduit. The depicted references 30, 31, 66 may be used to describe herein portions of one continuous fluid line or, in some implementations, may individually describe portions that are fluidly connected together to form a continuous fluid line. The upstream and downstream fluid lines and/or tube 30, 31, 66 may also be coupled to a pump cassette or cartridge that is configured to be coupled to the pump 10.

As shown in FIG. 1A, the face plate 50 which may be opened to reveal the internal loading mechanism for an infusion set. Within the housing of the infusion device (e.g., behind the door or face place), the infusion device includes a pumping segment including a group of serially-aligned pumping elements configured to compress an elongated compressible channel of an infusion set 66, when loaded within the pumping segment. The pumping segment includes a group of serially-aligned pumping elements (e.g., occluders and/or pumping finger(s)) configured to compress the elongated compressible channel (e.g., an IV tubing segment) loaded within the pumping segment.

The infusion set includes an intermediate section of the resiliently compressible tubing 66, for example of silicone rubber and, in some implementations, upper and/or lower fittings which each tubing section may be connected respectively with a respective upper line 30 and with the lower line 31. In use, each upper line 30 extends upwardly to a source of the medical fluid to be administered whilst the lower line 31 extends from the infusion pump to an infusion needle or the like inserted into the patient. In use, the infusion set 66 is extended across the face or deck of the pump unit so that its fittings (not labeled) are received in respective brackets respectively and so that the tubing segment extends over a pumping mechanism. In the depicted example, the pumping mechanism includes a four finger pump assembly 72, 74, 76, 78. In the depicted example, the infusion set is fitted in place in this fashion whilst the door 50 is in the open position. After the infusion line has been so fitted, the door 50 may be moved to the closed position and is secured by a catch 52 which may include a lever mounted on the outer edge of the door.

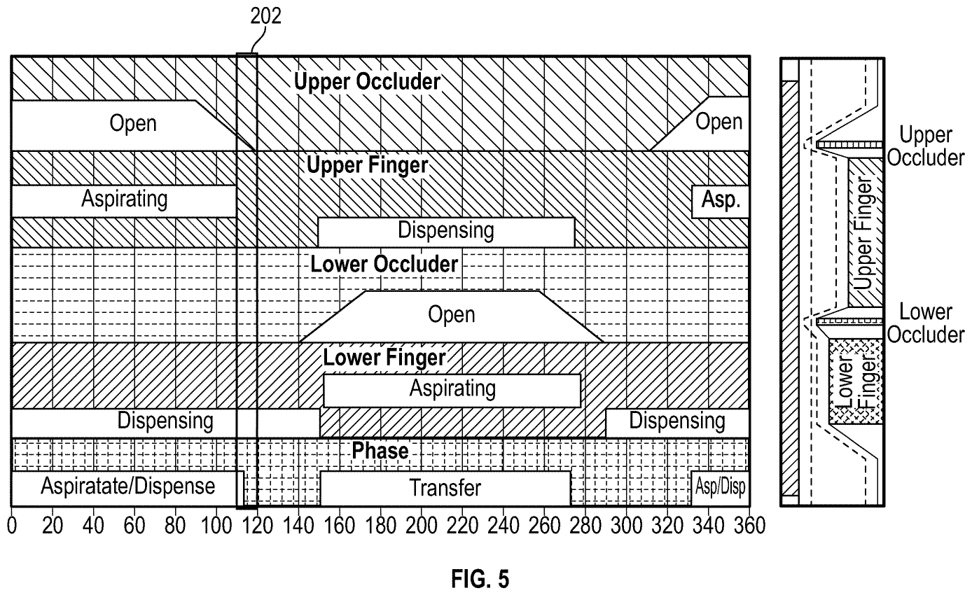

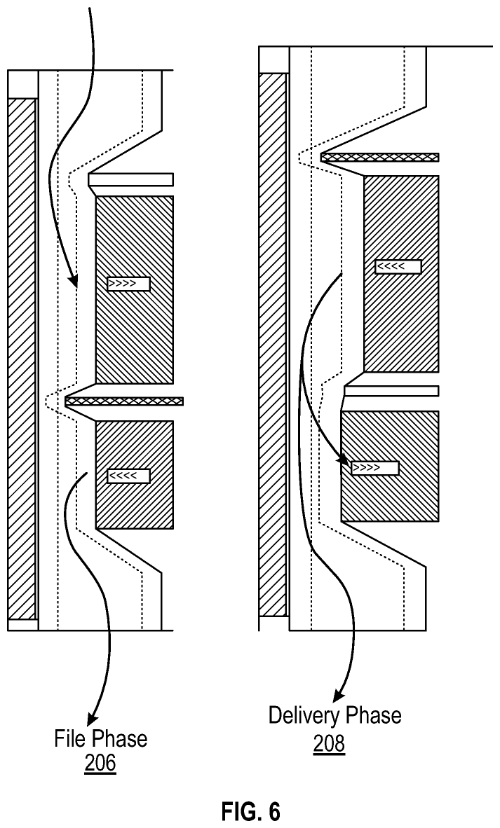

The four finger pump assembly 72, 74, 76, 78 includes respective fingers that are moveable by a cam system (See FIGS. 5 and 6) inwards and outwards from the face or deck of the pump to compress a respective tubing segment against a counter surface or anvil to propel fluid within the infusion line. In order to make it easier to maintain sterile conditions, these fingers may be covered by a thin flexible membrane, (not shown), sealed at its edges with respect to the deck. The fingers of the pump assembly periodically press the flexible resilient tubing against the counter surface which may be configured on an opposite side, for example, on an inner portion of the door 50.

The type of pumping mechanism may vary and may be for example, a multiple finger pumping mechanism. In the depicted example, the pumping mechanism includes an upstream occluding element or finger 72, a primary pumping element or finger 74, a downstream occluding element or finger 76, and a secondary pumping element or finger 78. The pumping mechanism (and mechanisms used in other linear peristaltic pumps) operate by sequentially pressing on a segment of the fluid conduit by means of the cam-following pumping elements (e.g., pumping fingers and valve fingers) 72, 74, 76, and 78, which in the depicted example make a four finger pump assembly. Each element may be sequentially activated by a respective cam lobe on a camshaft to apply a downward compression against tubing 66, to move the fluid in the tubing 66 downstream. Intermediate pumping mechanism 74 may include multiple intermediate elements or fingers (not shown) that sequentially activate according to positioning of the cam lobes. In some implementations, the pressure is applied in sequential locations of the conduit, beginning at the upstream end of the pumping mechanism, and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

An upstream pressure sensor 80 may also be included in the pump 10. The upstream pressure sensor is assigned to the flow control device or pumping mechanism 70 and, in this example, is further provided as an integral part of the infusion pump 10. It is mounted to the flow control device 70 and is located adjacent and upstream in relation to the flow control device. The upstream pressure sensor is located upstream from the flow control device, that is, at a location between a fluid supply and the flow control device, so that the connection of the correct fluid supply with the correct pump may be verified before any fluid is pumped to the patient. As will be described further, upstream pressure sensor 80 may be used to detect a pressure change adjacent the upstream occluder 72, to determine whether the occluder is functioning properly.

FIG. 1A further shows a downstream pressure sensor 82 included in the infusion pump 10 embodiment at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient and the flow control device, so that the connection of the correct fluid supply with the correct pump may be verified before any fluid is pumped to the patient. Downstream pressure sensor 82 may be used to detect a pressure change adjacent to occluder 76 and/or downstream finger 78, to determine whether these elements are functioning properly.

FIG. 1B depicts an example patient care unit 12 shown, according to various aspects of the subject technology. FIG. 1B shows two functional infusion pumps 10 (e.g., “infusion pump modules”) mounted at either side of a main frame infusion controller 14, and the displays and control keys of each, with the main frame infusion controller 14 being capable of programming both infusion pumps. The infusion device includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 10. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 10, such as alert indications (e.g., alarm messages). Control keys 5e-h may exist for programming and controlling operations of the infusion pump as desired. In some implementations, the control keys may be presented as interactive elements on the display 5c (e.g., touchscreen display). The main frame and/or functional module may also include audio alert equipment in the form of a speaker (not shown).

The main frame infusion controller 14 of the patient care unit 12 includes a display 6a for visually communicating various information, such as the operating parameters of a connected pump and alert indications and alert messages, and control keys 6b and 6c for selecting and/or setting control parameters and/or options for controlling the patient care unit 12 and connected modules. The main frame infusion controller 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The main frame infusion controller 14 may include a communications system (not shown) with which the main frame infusion controller 14 may communicate with external equipment such as a medical facility server or other computer and with a portable processor, such as a handheld communication device or a laptop-type of computer, or other information device that a clinician may have to transfer information as well as to download drug libraries to a functional module 10. The communication module may be used to transfer access and interaction information for clinicians encountering the main frame infusion controller or device coupled therewith (e.g., pump 10 or bar code scanner). The communications system may include one or more of a radio frequency (RF) system, an optical system such as infrared, a BLUETOOTH™M system, or other wired or wireless system. The bar code scanner and communications system may alternatively be included integrally with the infusion pump 10, such as in cases where a main frame infusion controller is not used, or in addition to one with the main frame infusion controller 14. Further, information input devices need not be hard-wired to medical instruments, information may be transferred through a wireless connection as well. Additionally, other types of modules may be connected to the pump modules or to the main frame infusion controller such as a syringe pump module, patient controlled analgesic module, end tidal CO₂ monitoring module, oximeter monitoring module, or the like.

FIG. 2 depicts an example pumping mechanism 120 of an infusion device 10 including two occluder valves 100, 110 (e.g., pumping elements 72, 76), according to various aspects of the subject technology. A typical medical pump for IV infusion delivery has two occluders, a first occluder 100 located upstream and a second occluder 110 located downstream, with a plunger 120 (e.g., pumping element 74) in between. The occluders and plunger coordinate with each other in programmable, sequential steps, controlled by a cam shaft to have two phases: 1) a filling phase, and 2) a delivery phase. The occluders move fluid in a tubing 103 by sequentially compressing the tubing, thereby causing a flow in a direction 104 according to the particular compression sequence of the occluders.

During the medication infusion process, in the filling phase, the upstream occluder 100 lifts to suck the medication into the tubing segment, which creates a pause, followed by the delivery phase to push the fluid out. These sequences can repeat through multiple cycles. To specify, when the plunger of a single plunger/tubing design is lifted from the tubing segment during the filling phase, there will be a disruption in the continuous infusion process.

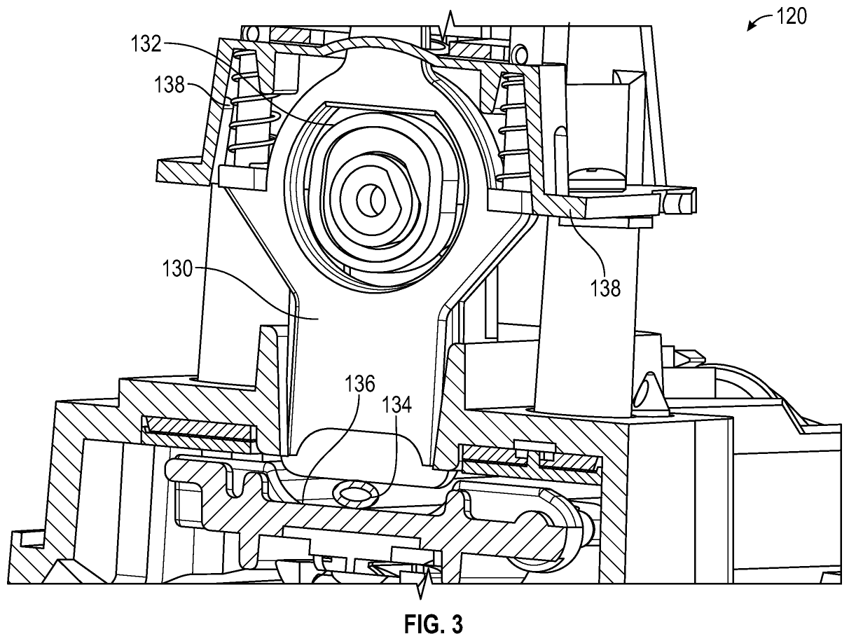

FIG. 3 depicts an example occluder valve 120, including an occluder element 130, according to various aspects of the subject technology. According to various implementations, there are two occluder elements 130; one for the upper occluder 72, and one for the lower occluder 76 of FIG. 1A. Occluder element 130 is configured to move according to a cam motion of a cam 132 to apply a periodic compression to a flexible infusion line 134 when the flexible infusion line is placed between the occluder element 130 and a plate assembly 136, also termed “platen”. In this regard, the cam motion oscillates the occluder element 130 to occlude a fluid within the flexible infusion line by periodically compressing the flexible infusion line. Compression springs 139 apply a constant force to the occluder element 130, forcing it against the plate assembly 136, while the cam 132 applies a force at predetermined intervals in an opposite direction, moving the lower portion of the occluder element responsible for compressing the infusion line 134 away from the plate assembly. Each cam 132 may be elliptical in shape and may rotate on an axis off center of the ellipse.

In the example of FIG. 3, the occluder element 130 is shown in the top dead center position (top position) with the stroke of the cam is fully upward and with the occluder element furthest apart from the plate assembly 136. A normal occluder element 130 compresses a tubing with a predetermined tolerance. That is, the occluder valve has a certain gap tolerance, or threshold distance between the platen 136 and the occluder element 130 when it is fully extended whereby the occluder element 130 will still fully compress the tubing 134. The platen 136 may be capable of moving towards and away with respect to the valve structure, and may be mechanically connected to the door mechanism of the door 50 shown in FIG. 1A. In this regard, the platen 136 may move away from the occluder element and an upper frame 139 of the valve 120 when the door 30 is opened, and move to lock in place as depicted in FIG. 3 when the door 50 is closed.

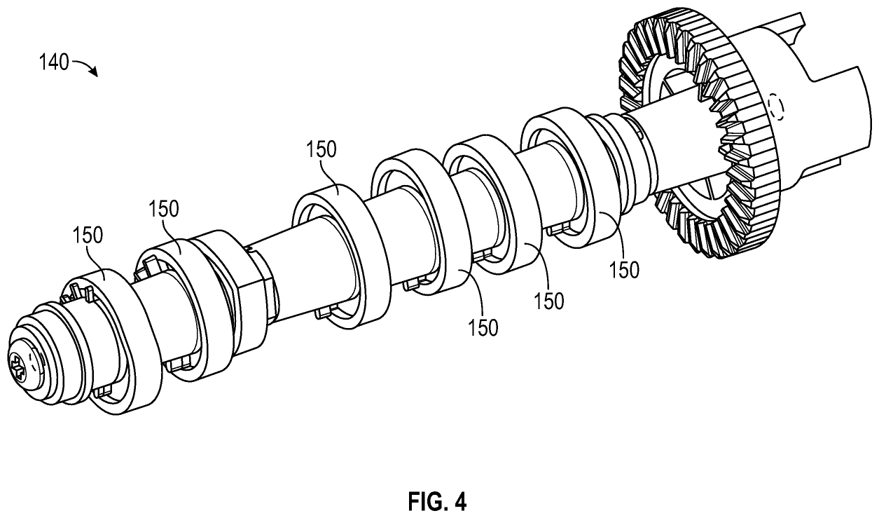

FIG. 4 depicts an example camshaft of a pumping element, according to various aspects of the subject technology. As described previously, upper and lower occluders 72, 76, and intermediate finger(s) 74 may each be movable by a cam 140. Integrally connected onto camshaft 140 at predetermined locations along the axis of the camshaft 140 may be a series of cam lobes 150. As will be appreciated by those skilled in the pertinent art, cam lobes 150 are eccentrically mounted on camshaft 140 in a pattern along the axis of camshaft 140 and are engaged with a respective pump element in a manner which, in some implementations, may create a wave-like movement of pumping elements when the camshaft 140 is rotated.

Once the fluid tubing of an infusion set 66 is loaded in infusion pump 10 and engaged with the four finger pump assembly, the door 50 is closed. The closure of door 50 causes the platen to contact a section of the tubing and enclose the section between the platen and a tube support (not shown) for supporting the tube in substantially a flat linear orientation. Activation of a stepper motor rotates a drive shaft causing a drive pulley to rotate the camshaft. The actual positioning of camshaft 140 may be represented by respective camshaft rotation angles θ, as depicted in FIG. 5. Revolution of camshaft 140 will cause the series of cam lobes 150 to reciprocate the pumping elements in a direction substantially perpendicular to the axis of, for example, the intermediate section of resiliently compressible tubing. Due to the configuration of cam lobes 150 on camshaft 140, the pumping elements may be sequentially urged against the tubing to create a moving zone of occlusion along the length the tubing during revolution of camshaft 140.

According to various implementations, the stepper motor assembly (not shown) and/or cam 140 of infusion pump 10 may be coupled to an encoder. In this manner, the encoder may encode the turns of the motor and/or the cam 140 for use in determining the current rotation angle of the cam 140. The encoder may transmit a single to the infusion pump's control unit (e.g., within main frame controller 14) which may, as described in further detail below, associate the current angle of the cam 140 with a pressure value.

FIG. 5 depicts an example cam phase diagram for testing a pumping element, according to various aspects of the subject technology. Each zone depicted in FIG. 5 corresponds to a pumping element 72, 74, 76, 78, and illustrates an example pumping function of the element according to a complete cam rotation (360°). That is which pumping elements are closing (compressing the tube) and which elements are opening, thereby creating an aspiration phase and a dispensing phase.

In the depicted example, initially, from 0° to 90°, the upper occluder 72 is open, while the lower occluder remains closed. The upper occluder 72 completes closing at about 120°; however, the fluid tubing may be sufficiently compressed to stop aspiration of the fluid at about 110°. The lower occluder remains closed until the cam reaches 140°. While the upper occluder 72 is open—until it begins to close at about 90°—the upper finger 74 is aspirating. The upper occluder closes between 90-120°, and the lower occluder begins to open at 140°. At this point, the lower finger will begin to deliver the fluid. In the depicted example, delivery begins at about 145°, with about 5° rotation accounting for the time to decompress the tubing.

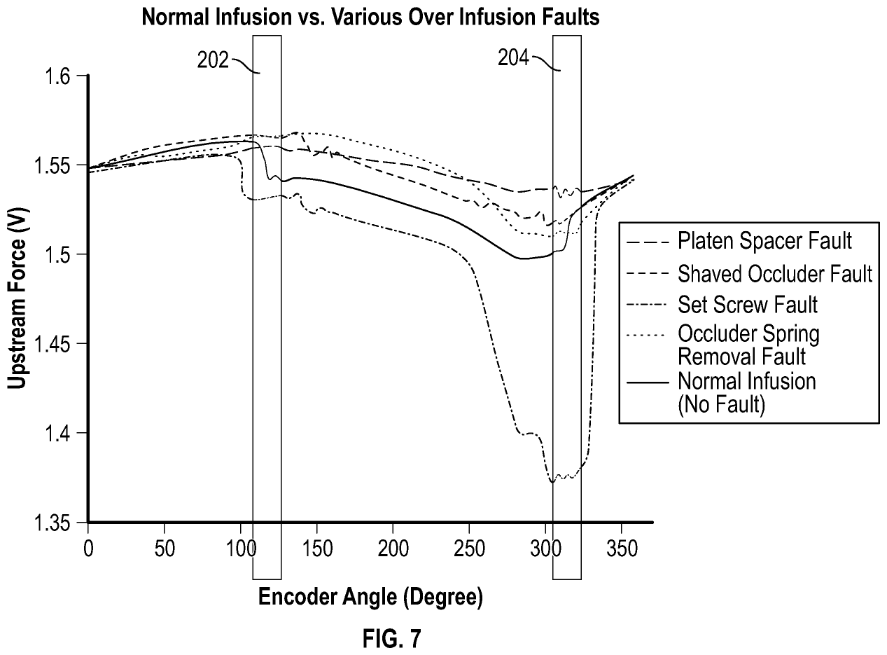

The stepper motor actuates the pumping finger which creates a signal in the line which is picked up by the pressure sensors 80, 82. Upper pressure sensor 80 signals are depicted in FIG. 7.

FIG. 6 depicts example fill and delivery phases, according to various aspects of the subject technology. In the depicted example, the infusion pump 10 is designed such that one occluder is always closed to prevent uncontrolled flow. With reference to FIGS. 4 and 5, the rotation of the cam 140 determines whether the pump is in the fill phase (206) or the delivery phase (208). During the fill phase (e.g., between a cam rotation angles of 312° and 112°) the lower occluder is closed and the upper occluder is open, letting the fluid from the bag into the pumping chamber. The secondary pumping finger pushes the fluid toward the patient. At the end of the fill phase (e.g., between the cam rotation angles of 112° and 312°), the upper occluder gets closed (i.e., both occluders are closed).

During the delivery phase (e.g., between cam rotation angles of 132° and 292°), the lower occluder gets opened and the primary pumping finger pushes the fluid toward the patient. At the end of the delivery phase (e.g., between the cam rotation angles of 292° and 132°), the lower occluder gets closed (i.e., both occluders are closed). Afterward, the upper occluder gets opened and the process repeats. Under normal conditions, free flow is not possible because one of the occluders is always engaged.

FIG. 7 depicts fault profiles in an upstream pressure response of a mechanically failing pumping mechanism, according to various aspects of the subject technology. During a pumping cycle (e.g., 360° cam rotation), upstream infusion line pressure may be continuously measured by sensor 80. A processor(s) (e.g., in main frame infusion controller 14 or infusion pump 10) analyzes pressure values measured by the upstream pressure sensor 80 over a portion of the pumping cycle. In the depicted example, values measured in an arc of rotation (202) between cam angles 105-120° are analyzed.

Under normal conditions, the pressure profile monitored by the sensor 80 will exhibit a known downward slope during the relevant arc of rotation (202), which is associated with application of at least one occluder and at least one pumping finger to the infusion line to move the fluid through the infusion line. The control unit determines a metric based on measuring the upstream pressure over the at least the portion of the pumping cycle and compares it to the known pressure profile(s). On a deviation between the determined metric and the known profile(s) corresponding to (e.g., being greater than) a deviation threshold, the control unit may determine a mechanical fault in the pumping mechanism has occurred.

In some implementations, the control unit determines a fault on detecting a predetermined drop (e.g., corresponding to a threshold) in pressure during the portion of the pumping cycle of the infusion pump. For example, the control unit may monitor a first portion of the cam cycle between 105° and 120° (202). In some implementations, the control unit determines a slope of measurement values obtained during the measuring of the upstream pressure over the at least the portion of the pumping cycle.

In some implementations, the control unit compares the determined slope with predetermined slopes corresponding to pump faults. For example, a first slope pattern may be representative of a platen spacer fault. A second slope pattern may be representative of a shaved occluder fault. A third slope pattern may be representative of a set screw fault, and a fourth slope pattern may be representative of an occluder spring removal fault. Based on matching the slopes, the control unit may identify a pump fault for the pumping cycle and provide an alert indicating the identified pump fault for display on a display device associated with the control unit. For example, the identified pump fault may be displayed on display 5c of the infusion pump 10, or displayed on display 6a of the main frame infusion controller 14. In some implementations, the identified pump fault may be provided to a server which may then provide the identified pump fault to a mobile device for display by the mobile device, remotely.

Additionally, or in the alternative, the control unit may monitor a second portion of the cam cycle (204). For example, the control unit may monitor an arc of rotation between 305° and 320°. In some implementations, the control unit may identify a pump fault based on matching metrics for both the first and second portions of to predetermined thresholds, as previously described.

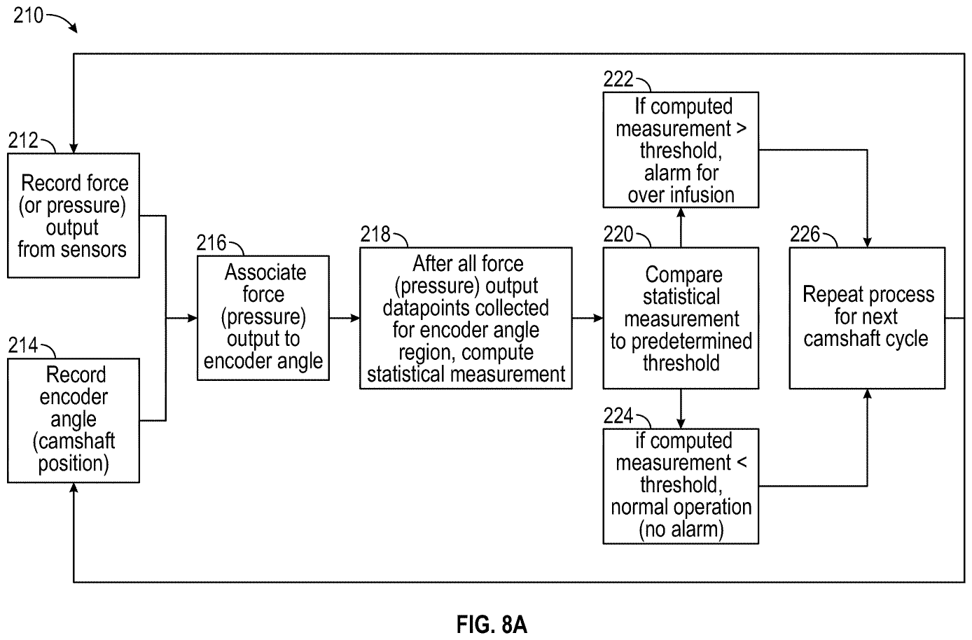

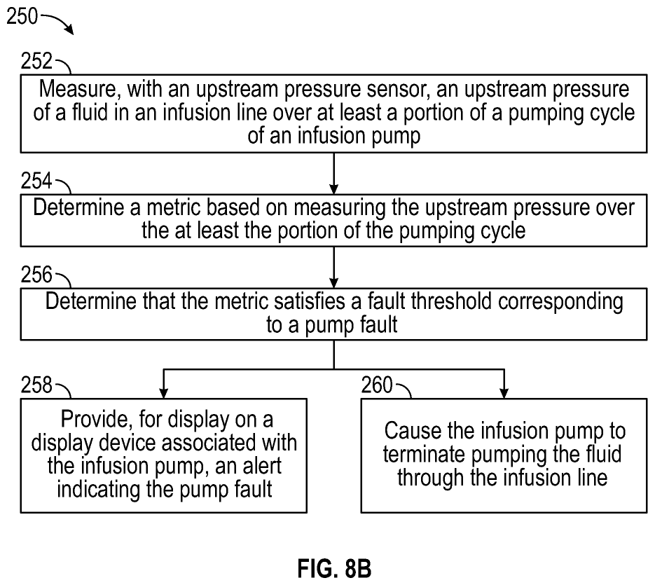

FIG. 8A depicts a first example process for testing a pumping element, according to aspects of the subject technology. A force pressure is measured in the infusion upstream from the pump by pump sensor 80 (212). The current angle of the cam is contemporaneously measured by an encoder (214). In some implementations, a pressure is measured for each angle. In some implementations, multiple pressure readings may be made for each angle, or readings may overlap angles. In some implementations, a pressure reading is measured for a predetermined range of angles.

The measured pressure is associated with an encoder angle (216). The control unit may then accumulate/collect pressure measurements/values over a predetermined arc of rotation. The measurements/values may then be transformed into a metric representative of a slope pattern. For example, the control unit may determine a statistical measurement for the measurement/values, such as a corresponding slope or waveform, mean or average value, or other statistical measurement pattern (218). The statistical measurement (e.g., waveform, slope, etc.) is then compared to a predetermined statistical threshold (220). The predetermined statistical threshold may be an average value or may take the form of the determined statistical measurement; e.g., a given slope or waveform, mean or average value, or other statistical measurement pattern.

If the determined measurement corresponds to the predetermined threshold (e.g., is greater than the threshold), the control unit may determine an alarm condition (222). According to various implementations, the alarm condition may include indicating that an over-infusion has occurred. According to various implementations, the alarm condition may include an indication of mechanical failure, and/or the type of mechanical failure, as described previously with regard to FIG. 7. If the determined measurement does not correspond to the predetermined threshold (e.g., is less than the threshold), the control unit may determine the infusion device is operating normally and that no alarm condition is warranted (224). The foregoing process (212-224) may then be repeated for each camshaft cycle (226).

In some implementations, the foregoing process (212-226) may be performed for datapoints/measurements collected for a relevant arc of rotation/encoder angle region. For example, as shown by the first encoder region in FIG. 7, the statistical measurement may be generated for an arc of rotation between 105° and 120°, as determined by the encoder. In another example, as shown by the second encoder region in FIG. 7, the statistical measurement may be generated for an arc of rotation between 305° and 320°. In some implementations, both regions may be combined in a single detection process. For example, the control unit may determine the statistical metric/measurement for each encoder region. An alert condition may then be determined when a selected one or both regions satisfy the corresponding respective threshold(s).

FIG. 8B depicts a second example process 250 for testing a pumping element, according to aspects of the subject technology. For explanatory purposes, the various blocks of example process 250 are described herein with reference to FIGS. 1 through 8A, and the components and/or processes described herein. The one or more of the blocks of process 250 may be implemented, for example, by one or more computing devices including, for example, within infusion device 10. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 250 are described as occurring in serial, or linearly. However, multiple blocks of example process 250 may occur in parallel. In addition, the blocks of example process 250 need not be performed in the order shown and/or one or more of the blocks of example process 250 need not be performed.

In the depicted example, at least one occluder element of an infusion pump 10 is activated to compress a fluid tubing filled with a fluid, to move the fluid in an upstream portion of the fluid tubing (e.g., in a fluid container upstream of the pump) to a downstream portion of the tubing, downstream of the infusion pump 10. As described previously, the infusion pump 10 includes or is associated with a control unit.

The control unit measures, using an upstream pressure sensor 80, an upstream pressure of a fluid in an infusion line over at least a portion of a pumping cycle of an infusion pump (252). As described previously with regard to FIGS. 6A and 6B, the pumping cycle includes application of the at least one occluder and at least one pumping finger to the infusion line to move the fluid through the infusion line. The control unit may receive an encoder signal from an encoder configured to monitor a pumping cam of the infusion device 10 and generate the encoder signal based on rotation of the pumping cam during the cam cycle. In this regard, a respective angle of rotation of the cam is determined based on the encoder signal.

The control unit determines a metric based on measuring the upstream pressure over the at least the portion of the pumping cycle (254). The metric may include, for example, a statistical measurement of the measurement/values, such as a corresponding slope or waveform, mean or average value, or other statistical measurement pattern. According to various implementations, the metric is determined for a partial rotation of a pumping cam in which a plurality of upstream pressure values are measured over a predetermined arc of rotation of the pumping cam, for example, corresponding to a fill phase, between a first predetermined angle of rotation and a second predetermined angle of rotation. A plurality of upstream pressure values may be measured over the cycle portion, and the metric determined (e.g., calculated based on the plurality of values.

In some implementations, the upstream pressure of the fluid may be measured over a first portion of the pumping cycle and a second portion of the pumping cycle. In some implementations, pressure data is collected over a plurality of cycles and the metric is determined based on values measured during the portion of the pumping cycle in each of the plurality of cycles.

The control unit determines that the metric corresponds to a fault threshold corresponding to a pump fault (256). In some implementations, the control unit may determine the pump fault by detecting a predetermined drop in pressure during the relevant portion (202, 204) of the pumping cycle. In some implementations, the control unit determines a slope (or other statistical measurement metric) of measurement values obtained during the measuring of the upstream pressure over the at least a portion of the pumping cycle and compares the slope with predetermined slopes corresponding to pump faults. In this manner, the control unit may identify a pump fault for the pumping cycle based on the comparing.

Responsive to determining that the determined metric satisfies the fault threshold, the control unit provides, for display on a display device associated with the infusion pump, an alert indicating the pump fault (258). Where a specific pump fault can be identified, the control unit may indicate the identified pump fault. Additionally, or in the alternative, responsive to determining that the metric satisfies the fault threshold, the control unit may cause the infusion pump 10 to terminate pumping the fluid through the infusion line, for example, by stopping the pump motor driving the camshaft. In some implementations, the speed of the motor may be reduced, thereby reducing the flow rate of the fluid. In this manner, an over-infusion may be prevented.

While detection of over-infusion is described herein as using pressure measurements from upstream pressure sensor 80, the detection may also use pressure measurements from downstream pressure sensor 82. In this regard, the predetermined fault profiles may be stored for occluder 76 and/or downstream finger 78. The control unit may measure, with pressure sensor 82, a downstream pressure of a fluid in the infusion line and determine a metric based the measured downstream pressure (measurements/values) over a portion of the pumping cycle, and determine that the metric satisfies a fault threshold corresponding to a pump fault.

According to various implementations, detection of an increase in pressure at a pressure sensor 80, 82 may indicate fault of the occluder element 72, 76 near the pressure sensor. In some implementations, an indication of the faulty occluder element may be provided for display by a display device, such as a display of the pump 10, or a computing device connected to the pump. For example, based on detecting the pressure increase by the upstream pressure sensor, an indication that the upper occluder element is faulty may be provided and displayed. Similarly, based on detecting the pressure increase by the downstream pressure sensor, an indication that the lower occluder element is faulty may be provided and displayed.

Many of the above-described devices, systems and methods, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

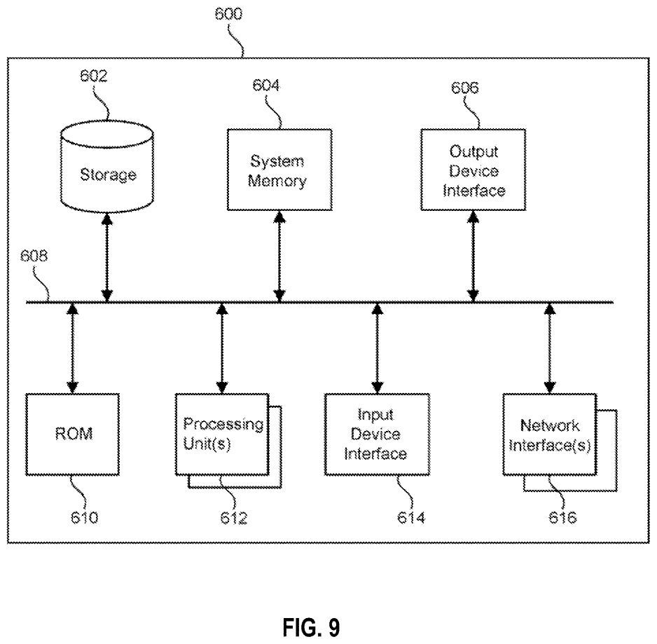

FIG. 9 is a conceptual diagram illustrating an example electronic system 600 for detecting an over-infusion of a fluid, according to aspects of the subject technology. Electronic system 600 may be a computing device for execution of software associated with one or more components and processes provided by FIGS. 1 through 8B, including but not limited to infusion pump 10 and/or a control unit associated with the infusion pump 10. Electronic system 600 may be representative of a device used in connection or combination with the disclosure regarding FIGS. 1 through 8B. In this regard, electronic system 600 may be a device connected to the infusion device 10, for example, to activate the occluders and/or pumping fingers, the cam 142, or to monitor or control same. For example, system 600 may be representative of a personal computer or a mobile device such as a smartphone, tablet computer, laptop, personal digital assistant (PDA), an augmented reality device, a wearable such as a watch or band or glasses, or combination thereof, or other touch screen or television with one or more processors embedded therein or coupled thereto, or any other sort of computer-related electronic device having network connectivity specifically configured to implement one or more of the features described.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves specific instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 9, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, a personal area network (“PAN”), or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of specific instructions for performing various operations described herein. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs) specifically configured with one or more of the features described. In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a specifically configured computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of specifically configured devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other (e.g., physically separated) and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A system for detecting an over-infusion of a fluid, comprising: an infusion pump; an pressure sensor disposed within the infusion pump to measure pressure of a fluid in an infusion line loaded into the infusion pump; and a control unit configured to: measure, over at least a portion of a pumping cycle of the infusion pump, with the pressure sensor, a pressure of the fluid in the infusion line loaded into the infusion pump, the pumping cycle including application of at least one occluder and at least one pumping finger to the infusion line to move the fluid through the infusion line; determine a metric based on measuring the pressure over the at least the portion of the pumping cycle; determine that the metric corresponds to a fault threshold associated with the at least the portion of the pumping cycle corresponding to an over-infusion fault; and provide, for display on a display device associated with the infusion pump, an alert indicating the over-infusion fault.
Clause 2. The system of Clause 1, wherein the control unit is further configured to: cause, responsive to determining that the metric corresponds to the fault threshold, the infusion pump to terminate or reduce pumping of the fluid through the infusion line.
Clause 3. The system of Clause 1 or Clause 2, wherein measuring the pressure over the at least the portion of the pumping cycle comprises measuring a plurality of pressure values over a fill phase, and wherein the metric is determined for the fill phase.
Clause 4. The system of any one of Clauses 1 through 3, further comprising: a pumping cam configured to operate at least one occluder valve and pumping mechanism of the infusion pump according to a rotation of the pumping cam, wherein measuring the pressure over the at least the portion of the pumping cycle comprises measuring a plurality of pressure values over a predetermined arc of rotation of the pumping cam, between a first predetermined angle of rotation and a second predetermined angle of rotation, in which a first occluder closes and a second occlude opens and a first pumping finger pushes the fluid through the infusion line.
Clause 5. The system of Clause 4, further comprising: an encoder configured to generate an encoder signal based on rotation of the pumping cam, wherein the control unit is further configured to: receive an encoder signal from an encoder configured to generate the encoder signal based on rotation of the pumping cam; and determine a respective angle of rotation of the pumping cam based on the encoder signal.
Clause 6. The system of Clause 5, wherein the control unit is further configured to: measure, with the pressure sensor, the pressure of the fluid over a first portion of the pumping cycle and a second portion of the pumping cycle.
Clause 7. The system of any one of Clauses 1 through 6, wherein the control unit is further configured to: determine a slope of measurement values obtained during the measuring of the pressure over the at least the portion of the pumping cycle; compare the slope with predetermined slopes corresponding to predetermined faults; identify a predetermined fault for the pumping cycle based on the comparing; and provide, for display on a display device associated with the control unit, an alert indicating the identified predetermined fault.
Clause 8. The system of any one of Clauses 1 through 7, wherein the control unit is further configured to: detect a predetermined drop in pressure during the portion of the pumping cycle of the infusion pump.
Clause 9. The system of any one of Clauses 1 through 8, wherein the control unit is further configured to: collect pressure data over a plurality of cycles and determining the metric based on values measured during the portion of the pumping cycle in each of the plurality of cycles.
Clause 10. A method for detecting an over-infusion of a fluid, comprising: measuring, over at least a portion of a pumping cycle of an infusion pump, with a pressure sensor, a pressure of the fluid in the infusion line loaded into the infusion pump, the pumping cycle including application of at least one occluder and at least one pumping finger to the infusion line to move the fluid through the infusion line; determining a metric based on measuring the pressure over the at least the portion of the pumping cycle; determining that the metric corresponds to a fault threshold associated with the at least the portion of the pumping cycle corresponding to an over-infusion fault; and providing, for display on a display device associated with the infusion pump, an alert indicating the over-infusion fault.
Clause 11. The method of Clause 10, further comprising: causing, responsive to determining that the metric satisfies the fault threshold, the infusion pump to terminate or reduce pumping of the fluid through the infusion line.
Clause 12. The method of Clause 10 or Clause 11, wherein measuring the pressure over the at least the portion of the pumping cycle comprises measuring a plurality of pressure values over a fill phase, and wherein the metric is determined for the fill phase.
Clause 13. The method of any one of Clauses 10 through 12, wherein measuring the pressure over the at least the portion of the pumping cycle comprises measuring a plurality of pressure values over a predetermined arc of rotation of a pumping cam of the infusion pump, between a first predetermined angle of rotation and a second predetermined angle of rotation, in which a first occluder closes and a second occlude opens and a first pumping finger pushes the fluid through the infusion line.
Clause 14. The method of Clause 13, further comprising: measuring, with the pressure sensor, the pressure of the fluid over a first portion of the pumping cycle and a second portion of the pumping cycle.
Clause 15. The method of Clause 13 or Clause 14, further comprising: receiving an encoder signal from an encoder configured to generate the encoder signal based on rotation of the pumping cam; and determining a respective angle of rotation of the pumping cam based on the encoder signal.
Clause 16. The method of any one of Clauses 10 through 15, further comprising: determining a slope of measurement values obtained during the measuring of the pressure over the at least the portion of the pumping cycle; comparing the slope with predetermined slopes corresponding to predetermined faults; identifying a predetermined fault for the pumping cycle based on the comparing; and providing, for display on a display device associated with the infusion pump, an alert indicating the identified predetermined fault.
Clause 17. The method of any one of Clauses 10 through 16, wherein measuring the pressure comprises: measuring the pressure using a pressure sensor integrated in the infusion pump.
Clause 18. The method of any one of Clauses 10 through 17, further comprising: detecting a predetermined drop in pressure during the portion of the pumping cycle of the infusion pump.
Clause 19. The method of any one of Clauses 10 through 18, further comprising: collecting pressure data over a plurality of cycles and determining the metric based on values measured during the portion of the pumping cycle in each of the plurality of cycles.
Clause 20. A non-transitory computer-readable medium having instructions stored thereon that, when executed by a computing device, cause the computing device to perform a method according to any one of Clauses 10 through 19.

### Further Consideration

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms, web page and server. As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

1. A system for detecting an over-infusion of a fluid, comprising:
an infusion pump;
an pressure sensor disposed within the infusion pump to measure pressure of a fluid in an infusion line loaded into the infusion pump; and
a control unit configured to:
measure, over at least a portion of a pumping cycle of the infusion pump, with the pressure sensor, a pressure of the fluid in the infusion line loaded into the infusion pump, the pumping cycle including application of at least one occluder and at least one pumping finger to the infusion line to move the fluid through the infusion line;
determine a metric based on measuring the pressure over the at least the portion of the pumping cycle;
determine that the metric corresponds to a fault threshold associated with the at least the portion of the pumping cycle corresponding to an over-infusion fault; and
provide, for display on a display device associated with the infusion pump, an alert indicating the over-infusion fault.

2. The system of claim 1, wherein the control unit is further configured to:
cause, responsive to determining that the metric corresponds to the fault threshold, the infusion pump to terminate or reduce pumping of the fluid through the infusion line.

3. The system of claim 1, or wherein measuring the pressure over the at least the portion of the pumping cycle comprises measuring a plurality of pressure values over a fill phase, and wherein the metric is determined for the fill phase.

4. The system claim 1, further comprising:
a pumping cam configured to operate at least one occluder valve and pumping mechanism of the infusion pump according to a rotation of the pumping cam,
wherein measuring the pressure over the at least the portion of the pumping cycle comprises measuring a plurality of pressure values over a predetermined arc of rotation of the pumping cam, between a first predetermined angle of rotation and a second predetermined angle of rotation, in which a first occluder closes and a second occlude opens and a first pumping finger pushes the fluid through the infusion line.

5. The system of claim 4, further comprising:
an encoder configured to generate an encoder signal based on rotation of the pumping cam, wherein the control unit is further configured to:
receive an encoder signal from an encoder configured to generate the encoder signal based on rotation of the pumping cam; and
determine a respective angle of rotation of the pumping cam based on the encoder signal.

6. The system of claim 5, wherein the control unit is further configured to:
measure, with the pressure sensor, the pressure of the fluid over a first portion of the pumping cycle and a second portion of the pumping cycle.

7. The system of claim 1, wherein the control unit is further configured to:
determine a slope of measurement values obtained during the measuring of the pressure over the at least the portion of the pumping cycle;
compare the slope with predetermined slopes corresponding to predetermined faults;
identify a predetermined fault for the pumping cycle based on the comparing; and
provide, for display on a display device associated with the control unit, an alert indicating the identified predetermined fault.

8. The system of claim 1, wherein the control unit is further configured to:
detect a predetermined drop in pressure during the portion of the pumping cycle of the infusion pump.

9. The system of claim 1, wherein the control unit is further configured to:
collect pressure data over a plurality of cycles and determining the metric based on values measured during the portion of the pumping cycle in each of the plurality of cycles.

10. A method for detecting an over-infusion of a fluid, comprising:
measuring, over at least a portion of a pumping cycle of an infusion pump, with a pressure sensor, a pressure of the fluid in the infusion line loaded into the infusion pump, the pumping cycle including application of at least one occluder and at least one pumping finger to the infusion line to move the fluid through the infusion line;
determining a metric based on measuring the pressure over the at least the portion of the pumping cycle;
determining that the metric corresponds to a fault threshold associated with the at least the portion of the pumping cycle corresponding to an over-infusion fault; and
providing, for display on a display device associated with the infusion pump, an alert indicating the over-infusion fault.

11. The method of claim 10, further comprising:
causing, responsive to determining that the metric satisfies the fault threshold, the infusion pump to terminate or reduce pumping of the fluid through the infusion line.

12. The method of claim 10, wherein measuring the pressure over the at least the portion of the pumping cycle comprises measuring a plurality of pressure values over a fill phase, and wherein the metric is determined for the fill phase.

13. The method of claim 10, wherein measuring the pressure over the at least the portion of the pumping cycle comprises measuring a plurality of pressure values over a predetermined arc of rotation of a pumping cam of the infusion pump, between a first predetermined angle of rotation and a second predetermined angle of rotation, in which a first occluder closes and a second occlude opens and a first pumping finger pushes the fluid through the infusion line.

14. The method of claim 13, further comprising:
measuring, with the pressure sensor, the pressure of the fluid over a first portion of the pumping cycle and a second portion of the pumping cycle.

15. The method of claim 10, further comprising:
receiving an encoder signal from an encoder configured to generate the encoder signal based on rotation of the pumping cam; and
determining a respective angle of rotation of the pumping cam based on the encoder signal.

16. The method of claim 10, further comprising:
determining a slope of measurement values obtained during the measuring of the pressure over the at least the portion of the pumping cycle;
comparing the slope with predetermined slopes corresponding to predetermined faults;
identifying a predetermined fault for the pumping cycle based on the comparing; and
providing, for display on a display device associated with the infusion pump, an alert indicating the identified predetermined fault.

17. The method of claim 10, wherein measuring the pressure comprises:
measuring the pressure using a pressure sensor integrated in the infusion pump.

18. The method of claim 10, further comprising:
detecting a predetermined drop in pressure during the portion of the pumping cycle of the infusion pump.

19. The method of claim 10, further comprising:
collecting pressure data over a plurality of cycles and determining the metric based on values measured during the portion of the pumping cycle in each of the plurality of cycles.

20. A non-transitory computer-readable medium having instructions stored thereon that, when executed by a computing device, cause the computing device to perform a method according, comprising:
measuring, over at least a portion of a pumping cycle of an infusion pump, with a pressure sensor, a pressure of the fluid in the infusion line loaded into the infusion pump, the pumping cycle including application of at least one occluder and at least one pumping finger to the infusion line to move the fluid through the infusion line;
determining a metric based on measuring the pressure over the at least the portion of the pumping cycle;
determining that the metric corresponds to a fault threshold associated with the at least the portion of the pumping cycle corresponding to an over-infusion fault; and
providing, for display on a display device associated with the infusion pump, an alert indicating the over-infusion fault.
